(19) United States
(12) Patent Application Publication
(10) Pub. No.: US 2003/0144541 A1
(43) Pub. Date: Jul. 31, 2003

(54) Process for preparing alpha-halogenated retones

(76) Inventor: Roland Jacquot, Francheville (FR)

Correspondence Address: Kevin McVeigh, Intellectual Property Department, Rhodia Inc CN 7500, 259 Prospect Plains Road, Cranbury, NJ, 08512-7500, US

(21) Appl. No.: 10/182,069

(22) PCT Filed: Jan. 26, 2001
(86) PCT No.: PCT/FR01/00256

(30) Foreign Application Priority Data
Jan. 27, 2000 (FR) 00/01051

(51) Int. Cl. C07C 45/29; C07C 253/30; C07C 225/16

(52) U.S. Cl. 558/414; 562/459; 564/502; 564/342; 568/322; 568/357

(57) ABSTRACT

The invention concerns a method for preparing α-halogenated ketones from secondary α-halogenated alcohols. More particularly, the invention concerns the preparation of α-trihalogenated ketones from secondary α-trihalogenated alcohols. The method for preparing said α-halogenated ketone is characterized in that it consists in oxidizing in liquid phase, a secondary α-halogenated alcohol, using molecular oxygen or a gas containing same, in the presence of a catalyst based on a metal M₁ selected among metals of group 1b and 8 of the periodic system of elements and optionally an activating element.

The present invention relates to a process for preparing α-halogenated ketones from α-halogenated secondary alcohols. More particularly, the invention relates to the preparation of α-trihalogenated ketones from α-trihalogenated secondary alcohols.

A α-trihalogenated ketone can be prepared using a process that consists of reacting an organometallic compound with trifluoroacetic acid or its esters [Chem. L. S. et al., J. Fluorine Chem. VIII, p. 117 (1981)].

That process suffers from a number of disadvantages. It comprises a plurality of steps, preparing an organometallic compound from bromobenzene then reacting with trifluoroacetic acid at low temperature (−78° C.) and hydrolysis, which complicates implementation, and makes it difficult to scale up.

Further, the reaction yield is not satisfactory due to the formation of by-products.

The aim of the present invention is to provide a novel process that can overcome those disadvantages.

We have now discovered, and this constitutes the subject matter of the present invention, a process for preparing an α-halogenated ketone, characterized in that it consists of oxidising, in the liquid phase, an α-halogenated secondary alcohol, using molecular oxygen or an oxygen-containing gas, in the presence of a catalyst based on a metal M₁ selected from metals from groups 1b and 8 of the periodic table.

In a preferred variation of the process of the invention, metals such as cadmium, cerium, bismuth, lead, silver, tellurium, tin or germanium are added as activators.

In one aspect, the invention provides a very generalised process for producing α-halogenated ketones from α-halogenated secondary alcohols with general formula (I):

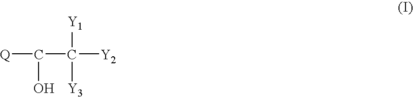

in which formula Q represents a monovalent hydrocarbon group, which may be substituted, containing 1 to 40 carbon atoms, Y₁, Y₂ and Y₃, which may be identical or different, represent a hydrogen atom or a halogen atom, namely chlorine, fluorine, bromine or iodine, preferably fluorine, or a perhalogenoalkyl group containing 1 to 10 carbon atoms, and at least one of groups Y₁, Y₂ and Y₃ represent a halogen atom.

Preferred compounds with formula (I) are those with formula (I) in which at least two of groups Y₁, Y₂ and Y₃ represent a halogen atom, more preferably all groups Y₁, Y₂ and Y₃ represent a halogen atom, preferably a fluorine atom.

The invention also envisages that the group CY₁Y₂Y₃ represents a perhalogenoalkyl group, preferably a perfluoroalkyl group, more preferably a trifluoromethyl group.

In formula (I), the group —CHOH—CY₁Y₂Y₃ is termed “halogenomethylcarbinol”.

The characteristic feature of the process of the invention is carrying out oxidation of α-halogenated secondary alcohols to the corresponding ketones in an aqueous or organic medium in the presence of a catalyst based on a metal M₁ selected from metals from groups 1b and 8, and an optional activator.

A definition of the metallic elements can be found in the periodic table published in the “Bulletin de la Societe Chimique de France”, n° 1 (1966).

More precisely, the α-halogenated secondary alcohols acting as starting substances for the preparation of ketones have general formula (I) in which Q represents a monovalent hydrocarbon group, which may or may not be substituted, which may be a linear or branched, saturated or unsaturated acyclic aliphatic group; or a saturated, unsaturated or aromatic, monocyclic or polycyclic, carbocyclic or heterocyclic group.

Particular suitable α-halogenated secondary alcohols with general formula (I) for use in the process of the invention are those in which Q represents a monocyclic or polycyclic aromatic hydrocarbon group; the groups can between them form ortho-condensed systems (for example the naphthyl group) or ortho- and peri-condensed systems.

Preferably, Q represents an aryl group with general formula (II):

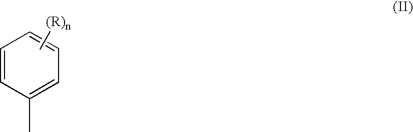

in which formula (II):

n is a whole number from 0 to 5, preferably 0 to 3;

R represents R₁, one of the following groups or functions:

a linear or branched alkyl group containing 1 to 6 carbon atoms, preferably 1 to 4 carbon atoms, such as methyl, ethyl, propyl, isopropyl, butyl, isobutyl, sec-butyl or tert-butyl;

a halogenoalkyl group containing 1 to 6 carbon atoms, which may be mono-, poly- or per-halogenoalkyl, containing 1 to 13 halogen atoms;

a linear or branched alkenyl group containing 2 to 6 carbon atoms, preferably 2 to 4 carbon atoms, such as vinyl, allyl;

a linear or branched alkoxy or thioether group containing 1 to 6 carbon atoms, preferably 1 to 4 carbon atoms, such as methoxy, ethoxy, propoxy, isopropoxy or butoxy;

a group with formula:

—OH

—COOH

—CHO

—CN

—N—(R₂)₂

—X

—CF₃

in which formulae groups R₂, which may be identical or different, represent a hydrogen atom, a linear or branched alkyl group containing 1 to 6 carbon atoms, more preferably 1 to 4 carbon atoms, or a phenyl group, and X represents a halogen atom, in particular a chlorine or bromine atom;

R represents R₃, one of the following more complex groups:

a group:

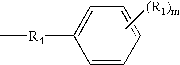

in which:

R₁ has the meanings given above;

R₄ represents a covalent bond or a linear or branched, saturated or unsaturated divalent hydrocarbon group containing 1 to 4 carbon atoms, such as methylene, ethylene, propylene, isopropylene or isopropylidene;

and m is a whole number from 0 to 3;

a group R₄—A—R₅, in which:

R₄ has the meanings given above;

R₅ represents a linear or branched alkyl group containing 1 to 6 carbon atoms, preferably 1 to 4 carbon atoms, or a group:

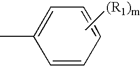

and A represents one of the following groups:

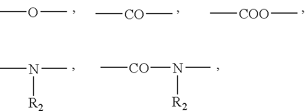

in which formulae m, R₁ and R₂ have the meanings given above.

When n is more than 1, groups R can be identical or different and 2 successive carbon atoms on the benzene ring can be bonded together via a ketal bridge such as extranuclear methylenedioxy or ethylenedioxy groups.

Preferably, n is 0, 1, 2 or 3.

Of all of groups Q cited above, preferred α-halogenated secondary alcohols for use in the process of the invention are those with general formula (I) in which Q represents an aryl group with general formula (II) in which:

n is 0, 1, 2 or 3;

R represents one of the following groups or functions:

a linear or branched alkyl group containing 1 to 4 carbon atoms;

a linear or branched alkoxy or thioether group containing 1 to 4 carbon atoms;

a methylene or ethylenedioxy group;

an —OH group.

Examples of α-halogenated secondary alcohols with general formula (I) in which Q represents an aryl group with general formula (II) that can be cited are: 2-hydroxy-1-phenyl-trichloromethylcarbinol; 2-hydroxy-1-phenyl-trifluoromethylcarbinol; 3-hydroxy-1-phenyl-trichloromethylcarbinol; 3-hydroxy-1-phenyl-trifluoromethylcarbinol; : 4-hydroxy-1-phenyl-trichloromethylcarbinol; 4-hydroxy-1-phenyl-trifluoromethylcarbinol; : 2-hydroxy-3-methyl-1-phenyl-trichloromethylcarbinol; 2-hydroxy-3-methyl-1-phenyl-trifluoromethylcarbinol; 2-hydroxy-4-methyl-1-phenyl-trichloromethylcarbinol; 2-hydroxy-4-methyl-1-phenyl-trifluoromethylcarbinol; 2-hydroxy-5-methyl-1-phenyl-trichloromethylcarbinol; 2-hydroxy-5—methyl-1-phenyl-trifluoromethylcarbinol; 3-hydroxy-4-methyl-1-phenyl-trichloromethylcarbinol; 3-hydroxy-4-methyl-1-phenyl-trifluoromethylcarbinol; 2-methoxy-1-phenyl-trichloromethylcarbinol; 2-methoxy-1-phenyl-trifluoromethylcarbinol; 3-methoxy-1-phenyl-trichloromethylcarbinol; 3-methoxy-1-phenyl-trifluoromethylcarbinol; 4-methoxy-1-phenyl-trichloromethylcarbinol; 4-methoxy-1-phenyl-trifluoromethylcarbinol; 5-methoxy-1-phenyl-trichloromethylcarbinol; 5-methoxy-1-phenyl-trifluoromethylcarbinol; 3-hydroxy-4-methoxy-1-phenyl-trichloromethylcarbinol; 3-hydroxy-4-methoxy-1-phenyl-trifluoromethylcarbinol; 4-hydroxy-3-methoxy-1-phenyl-trichloromethylcarbinol; 4-hydroxy-3-methoxy-1-phenyl-trifluoromethylcarbinol; 3-hydroxy-4,5-dimethoxy-1-phenyl-trichloromethylcarbinol; 3-hydroxy-4,5-dimethoxy-1-phenyl-trifluoromethylcarbinol; 4-hydroxy-3,5-dimethoxy-1-phenyl-trichloromethylcarbinol; 4-hydroxy-3,5-dimethoxy-1-phenyl-trifluoromethylcarbinol; 5-hydroxy-1-phenyl-3-bis(trichloromethylcarbinol); 5-hydroxy-1-phenyl-3-bis(trifluoromethylcarbinol); 4-hydroxy-3,5-dimethoxy-1-phenyl-trichloromethylcarbinol; 4-hydroxy-3,5-dimethoxy-1-phenyl-trifluoromethylcarbinol; 2-hydroxy-3-amino-1-phenyl-trichloromethylcarbinol; 2-hydroxy-3-amino-1-phenyl-trifluoromethylcarbinol; 2-hydroxy-4-amino-1-phenyl-trichloromethylcarbinol; 2-hydroxy-4-amino-1-phenyl-trifluoromethylcarbinol; 2-hydroxy-5-amino-1-phenyl-trichloromethylcarbinol; 2-hydroxy-5-amino-1-phenyl-trifluoromethylcarbinol; 3-hydroxy-2-amino-1-phenyl-trichloromethylcarbinol; 3-hydroxy-2-amino-1-phenyl-trifluoromethylcarbinol; 2,3-dihydroxy-1-phenyl-trichloromethylcarbinol; 2,3-dihydroxy-1-phenyl-trifluoromethylcarbinol; 2,4-dihydroxy-1-phenyl-trichloromethylcarbinol; 2,4-dihydroxy-1-phenyl-trifluoromethylcarbinol; 2,5-dihydroxy-1-phenyl-trichloromethylcarbinol; 2,5-dihydroxy-1-phenyl-trifluoromethylcarbinol; 2,6-dihydroxy-1-phenyl-trichloromethylcarbinol; 2,6-dihydroxy-1-phenyl-trifluoromethylcarbinol; 3,4-dihydroxy-1-phenyl-trichloromethylcarbinol; 3,4-dihydroxy-1-phenyl-trifluoromethylcarbinol; 3,5-dihydroxy-1-phenyl-trichloromethylcarbinol; 3,5-dihydroxy-1-phenyl-trifluoromethylcarbinol; 3,5-dihydroxy-4-methyl-1-phenyl-trichloromethylcarbinol; 3,5-dihydroxy-4-methyl-1-phenyl-trifluoromethylcarbinol; 2,3,4-trihydroxy-1-phenyl-trichloromethylcarbinol; 2,3,4-trihydroxy-1-phenyl-trifluoromethylcarbinol; 2,4,6-trihydroxy-1-phenyl-trichloromethylcarbinol; 2,4,6-trihydroxy-1-phenyl-trifluoromethylcarbinol; 3,4,5-trihydroxy-1-phenyl-trichloromethylcarbinol; 3,4,5-trihydroxy-1-phenyl-trifluoromethylcarbinol.

In general formula (I) for α-halogenated secondary alcohols, Q can represent a carbocyclic group that is saturated or comprises 1 or 2 unsaturated bonds in the cycle, generally containing 3 to 7 carbon atoms, preferably 6 carbon atoms in the cycle; said cycle can be substituted by 1 to 5 groups R₁, preferably 1 to 3, R₁ having the meanings given above for the aryl substituents with general formula (II).

Preferred examples of groups Q that can be cited are cyclohexyl or cyclohexene-yl groups, optionally substituted with linear or branched alkyl groups containing 1 to 4 carbon atoms.

Examples of α-halogenated secondary alcohols with formula (I) in which Q is a cycloaliphatic group that can be cited are 1-(trichloromethylcarbinol)-1-cyclohexene, 1-(trifluoromethylcarbinol)-1-cyclohexene, 1-(trichloromethylcarbinol)-1-cyclohexane, 1-(trifluoromethylcarbinol)-1-cyclohexane, 1-methyl-2-(trichloromethylcarbinol)-1-cyclohexene, 1-methyl-2-(trifluoromethylcarbinol)-1-cyclohexene, 1-methyl-2-(trichloromethylcarbinol)-cyclohexane, 1-methyl-2-(trifluoromethylcarbinol)-cyclohexane, 1-methyl-4-isopropyl-2-(trichloromethylcarbinol)-1-cyclohexene, 1-methyl-4-isopropyl-2-(trifluoromethylcarbinol)-1-cyclohexene, 1-methyl-4-isopropyl-(trichloromethylcarbinol)-1-cyclohexane, 1-methyl-4-isopropyl-(trifluoromethylcarbinol)-cyclohexane.

As mentioned above, Q can represent a linear or branched, saturated or unsaturated acyclic aliphatic group.

More precisely, Q represents a linear or branched alkyl, alkenyl, alkadienyl or alkynyl group, preferably containing 1 to 12 carbon atoms.

The hydrocarbon chain can optionally be:

interrupted by one of the following groups:

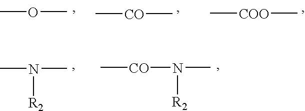

in which formulae R₂ has the meaning given above;

and/or carries one of the following substituents:

—OH, —COOH, —CHO, —CN, —N(R₂)₂, —X, —CF₃

in which formulae R₂ has the meaning given above.

The linear or branched, saturated or unsaturated acyclic aliphatic group can optionally carry a cyclic substituent. The term “cycle” means a saturated, unsaturated or aromatic carbocyclic or heterocyclic cycle.

The acyclic aliphatic group can be bonded to the cycle via a covalent bond or by one of the following groups:

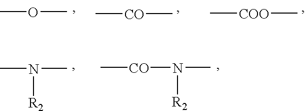

in which formula R₂ has the meaning given above.

Examples of cyclic substituents that can be envisaged are cycloaliphatic, aromatic or heterocyclic substituents, in particular cycloaliphatics containing 6 carbon atoms in their cycle or benzene rings, said cyclic substituents themselves optionally carrying 1, 2, 3, 4 or 5 groups R₁, which may be identical or different, R₁ having the meaning given above.

Examples of α-halogenated secondary alcohols with formula (I) in which Q represents an aliphatic group that can be cited are: 1,1,1-trifluoro-2-pentanol, 4-methyl-1,1,1-trichloro-2-pentanol, 1,1,1-trifluoro-2-hexanol, 3,3-dimethyl-1,1,1-trifluoro-2-butanol, 2-hydroxy-4-methoxy-1,1,1-trichloro-5-pentanol, 1,1,1-trichloro-2-heptanol, 5-hydroxy-4-methyl-6,6,6-trichloro-3-hexanone, 2-hydroxy-1,1,1-trichloro-4-octanone, 2-hydroxy-6-methyl-1,1,1-trichloro-4-heptanone, 4-ethyl-1,1,1-trichloro-2-hexanol, 3-ethyl-1,1,1-trichloro-2-heptanol, 2-hydroxy-1,1,1-trichloro-4-nonanone, 2-hydroxy-7-methyl-1,1,1-trichloro-4-octanone, 1,1,1-trichloro-4,6,6-trimethyl-2-heptanol, 1,1,1-trichloro-2-nonanol, 2-hydroxy-1,1,1-trichloro-4-decanone, 2-hydroxy-1,1,1-trichloro-4-undecanone, 1,1,1-trichloro-2-dodecanol, 1,1,1-trichloro-3-pentene-2-ol, 1,1,1-trifluoro-3-pentene-2-ol, 3-methyl-1,1,1-trichloro-3-butene-2-ol, 5,5,5-trichloro-1-pentene-4-ol, 4-methyl-1,1,1-trichloro-3-pentene-2-ol, 3-methyl-1,1,1-trichloro-3-pentene-2-ol, 4-methyl-1,1,1-trifluoro-3-pentene-2-ol, 3,4-dimethyl-1,1,1-trichloro-3-pentene-2-ol, 4-ethyl-1,1,1-trichloro-3-hexene-2-ol; 1,1,1-trifluoro-4-hexene-2-ol, 1,1,1-trichloro-3-nonene-2-ol, 1,1,1,4-tetrachloro-3-nonene-2-ol, 1,1,1-trichloro-3-dodecene-2-ol, 1,1,1-trifluoro-4-octene-2-ol, 7-bromo-1,1,1-trichloro-7-octene-3-yne-2-ol, 8-bromo-1,1,1-trichloro-7-octene-3-yne-2-ol, 1,1,1-trichloro-3-nonyne-2-ol, 1,1,1-trichloro-3-decyne-2-ol, 1,1,1-trichloro-4-(4-methyl-3-cyclohexene-1-yl)-3-pentene-2-ol; 9-trichloroethylol limonene, (3,4,5,6-tetrahydro)-4-nonatolyl-1,1,1-trichloro-2-pentanol, 4-phenyl-1,1,1-trichloro-3-pentene-2-ol, 4-phenyl-1,1,1-trichloro-2-pentanol, 4,6,6-trimethyl-1,1,1-trichloro-3-heptene-2-ol, 4,6,6-trimethyl-1,1,1-trichloro-2-heptanol, 5-methyl-1,1,1-trichloro-5-hexene-2-ol, 5-methyl-1,1,1-trichloro-2-hexanol, 3,4-dimethyl-1,1,1-trichloro-2-pentanol.

Q can also represent a saturated or non saturated heterocylic group, in particular containing 5 or 6 atoms in the cycle, 1 or 2 of which are heteroatoms such as nitrogen, sulphur or oxygen, the carbon atoms of the heterocycle possibly being substituted, either completely or only partially by groups R₁, R₁ having the meanings given above for the substituents on the aryl group with formula (II).

Q can also represent a polycyclic heterocyclic group defined as either a group constituted by at least two aromatic or non aromatic heterocycles containing at least one heteroatom in each cycle and between them forming ortho- or ortho- and peri-condensed systems, or a group constituted by at least one aromatic or non aromatic hydrocarbon cycle and at least one aromatic or non aromatic heterocycle forming ortho- or ortho- and peri-condensed systems between them.

Examples of α-halogenated secondary alcohols with formula (I) in which Q represents a heterocyclic group that can be cited are 2-furyl-trichloromethylcarbinol, 2-furyl-trifluoromethylcarbinol, 1-(5-methylfuryl)-trichloromethylcarbinol, 1-(5-N,N-diethylfuramide)-trichloromethylcarbinol, (2,2,2-trifluoro-1-ethanol)-3-pyridine, 2-amino-4-hydroxy-6-methyl-5-(trichloromethylcarbinol)-pyrimidine, 2-amino-4-hydroxy-6-methyl-5-(trifluoromethylcarbinol)-pyrimidine, 4-hydroxy-6-methyl-2-methylamino-5-(2,2,2-trichloro-1-hydroxyethyl)-pyrimidine, 2-dimethylamino-4-hydroxy-6-methyl-5-(2,2,2-trichloro-1-hydroxyethyl)-pyrimidine.

Starting α-halogenated secondary alcohols that can be oxidised to ketones using the process of the invention are obtained by processes described in the literature. In particular, they can be prepared using one or other of the preparation procedures cited by J. H. T. LEDRUT and G. COMBES in “Industrie chimique beige” n° 6 (1962), p. 635 to 652.

Of the various processes for producing α-halogenated secondary alcohols, certain thereof are more suitable than others for preparing certain families of this type of compound.

Thus, for example, compounds with a labile hydrogen can react with chloral (or bromal) to produce the corresponding α-halogenated secondary alcohol.

It is possible to use an acid catalyst such as aluminium chloride to react aromatic hydrocarbons such as veratrole (or 1,2-dimethoxybenzene) with chloral. For this type of preparation, reference should be made, in addition to the article cited above, to the article by R. QUELET in the Bulletin de la Société Chimique de France (1954), p. 93².

When starting from phenols, it is possible to react chloral in the presence of anhydrous potassium carbonate (see M. PAULY, Berichte der Deutschen Gesellschaft 56, 979 (1923)).

It is also possible to use α-halogenated secondary alcohols prepared in accordance with the Applicant's PCT applications PCT/FR99/01235 and PCT/FR99/01379.

The catalysts used in the process of the invention are based on a metal from groups 1b and 8 of the periodic table.

Examples of catalysts based on a metal from group 8 of the periodic table that can be cited are nickel and noble metals such as ruthenium, rhodium, palladium, osmium, iridium, platinum and mixtures thereof. A preferred metal from group 1b is copper.

Preferably, platinum and/or palladium catalysts are used, employed in any available form such as: platinum black, palladium black, platinum oxide, palladium oxide or the noble metal itself deposited on various supports such as carbon black, calcium carbonate, activated alumina and silica or similar substances. Catalytic masses based on carbon black are particularly suitable.

The quantity of this catalyst to be used, expressed as the weight of metal M₁ with respect to that of the compound with formula (I), can be between 0.01% and 10%, preferably 0.04% to 2%.

Reference should be made to U.S. Pat. No. 3,673,257 and French patents FR-A-2 305 420 and FR-A-2 350 323 for further details regarding the catalysts.

The activator can be selected from any of those mentioned in the patents cited above. Preferably, bismuth, lead or cadmium are used, in the form of the free metals or cations. In the latter case, the associated anion is not critical and any derivatives of these metals can be used. Preferably, bismuth metal or its derivatives is used.

An inorganic or organic bismuth derivative in which the bismuth atom has an oxidation number greater than zero, for example 2, 3, 4 or 5, can be used. The residue associated with the bismuth is not critical if it satisfies this condition. The activator can be soluble or insoluble in the reaction medium.

Examples of activators suitable for use in the process of the present invention are: bismuth oxides; bismuth hydroxides; salts of mineral hydrogen acids such as: bismuth chloride, bromide, iodide, sulphide, selenide or telluride; salts of mineral oxyacids such as: bismuth sulphite, sulphate, nitrite, nitrate, phosphite, phosphate, pyrophosphate, carbonate, perchlorate, antimonate, arsenate, selenite or selenate; salts of oxyacids derived from transition metals, such as: bismuth vanadate, niobate, tantalate, chromate, molybdate, tungstate or permanganate.

Other suitable compounds are salts of organic aliphatic or aromatic acids, such as: bismuth acetate, propionate, benzoate, salicylate, oxalate, tartrate, lactate or citrate; and phenates such as: bismuth gallate or pyrogallate. These salts and phenates may also be bismuthyl salts.

Other inorganic or organic compounds that can be cited are binary combinations of bismuth with elements such as phosphorus and arsenic; heteropolyacids containing bismuth and their salts; also aliphatic or aromatic bismuthines.

Specific examples that can be cited are:

oxides: BiO; Bi₂O₃; Bi₂O₄; Bi₂O₅,

hydroxides: Bi(OH)₃,

salts of mineral hydrogen acids: bismuth chloride BiCl₃; bismuth bromide BiBr₃; bismuth iodide BiI₃; bismuth sulphide Bi₂S₃; bismuth selenide Bi₂Se₃; bismuth telluride Bi₂Te₃,

salts of mineral oxyacids: basic bismuth sulphite Bi₂(SO₃)₃,Bi₂O₃,5H₂O; neutral bismuth sulphate Bi₂(SO₄)₃; bismuthyl sulphate (BiO)HSO₄; bismuthyl nitrite (BiO)NO₂,0.5H₂O; neutral bismuth nitrate Bi(NO₃)₃,5H₂O; the double nitrate of bismuth and magnesium 2Bi(NO₃)₃,3Mg(NO₃)₂,24H₂O; bismuthyl nitrate (BiO)NO₃; bismuth phosphite Bi₂(PO₃H)₃, 3H₂O; neutral bismuth phosphate BiPO₄; bismuth pyrophosphate Bi₄(P₂O₇)₃; bismuthyl carbonate (BiO)₂CO₃,0.5H₂O; neutral bismuth perchlorate Bi(ClO₄)₃,5H₂O; bismuthyl perchlorate (BiO)ClO₄; bismuth antimonate BiSbO₄; neutral bismuth arsenate Bi(AsO₄)₃; bismuthyl arsenate (BiO)AsO₄,5H₂O; bismuth selenite Bi₂(SeO₃)₃,

salts of oxyacids derived from transition metals: bismuth vanadate BiVO₄; bismuth niobate BiNbO₄; bismuth tantalate BiTaO₄; neutral bismuth chromate Bi₂(CrO₄); bismuthyl dichromate [(BiO)₂]₂Cr₂O₇; acidic bismuthyl chromate H(BiO)CrO₄; the double chromate of bismuthyl and potassium K(BiO)CrO₄; bismuth molybdate Bi₂(MoO₄)₃; bismuth tungstate Bi₂(WO₄)₃; the double molybdate of bismuth and sodium NaBi(MoO₄)₂; basic bismuth permanganate Bi₂O₂(OH)MnO₄,

salts of organic aliphatic or aromatic acids: bismuth acetate Bi(C₂H₃O₂)₃; bismuthyl propionate (BiO)C₃H₅O₂; basic bismuth benzoate C₆H₅CO₂Bi(OH)₂; bismuthyl salicylate C₆H₄CO₂(BiO)(OH); bismuth oxalate (C₂O₄)₃Bi₂; bismuth tartrate Bi₂(C₄H₄O₆)₃,6H₂O; bismuth lactate (C₆H₉O₅)OBi, 7H₂O; bismuth citrate C₆H₅O₇Bi,

phenates: basic bismuth gallate C₇H₇O₇Bi; basic bismuth pyrogallate C₆H₃(OH)₂(OBi)(OH).

Other suitable inorganic or organic compounds are: bismuth phosphide BiP; bismuth arsenide Bi₃As₄; sodium bismuthate NaBiO₃; bismuth-thiocyanic acids H₂[Bi(BNS)₅], H₃[Bi(CNS)₆] and their sodium and potassium salts; trimethylbismuthine Bi(CH₃)₃, and triphenylbismuthine Bi(C₆H₅)₃.

Preferred bismuth derivatives for use in the process of the invention are: bismuth oxides; bismuth hydroxides; the bismuth or bismuthyl salts of mineral hydrogen acids; the bismuth or bismuthyl salts of mineral oxyacids; the bismuth or bismuthyl salts of organic aliphatic or aromatic acids; and bismuth or bismuthyl phenates.

A particularly suitable group of activators for use in the process of the invention is formed by: bismuth oxides Bi₂O₃ and Bi₂O₄; bismuth hydroxide Bi(OH)₃; neutral bismuth sulphate Bi₂(SO₄)₃; bismuth chloride BiC1₃; bismuth bromide BiBr₃; bismuth iodide BiI₃; neutral bismuth nitrate Bi(NO₃)₃,5H₂O; bismuthyl nitrate BiO(NO₃); bismuthyl carbonate (BiO)₂CO₃,0.5H₂O; bismuth acetate Bi(C₂H₃O₂)₃; bismuthyl salicylate C₆H₄CO₂(BiO)(OH).

The quantity of activator used, expressed as the quantity of metal contained in the activator with respect to the weight of metal M₁ employed, can vary between wide limits. This quantity can be as low as 0.1%, for example, and can also equal or exceed 10% of the weight of the metal M₁ employed without detriment. It is advantageously about 50%.

Depending on the nature of the starting substrate, the process of the invention has a number of implementations.

If the starting α-halogenated secondary alcohol has formula (I) in which Q is an aryl group and carries at least one hydroxyl group, it is advantageous to react that compound with a phenol type compound in the salt form.

In that case, the catalytic entity may or may not be formed in situ by successive or simultaneously introducing the catalyst based on metal M₁ and the activator.

If the starting α-halogenated secondary alcohol is not a phenol type compound, the oxidation reaction can be carried out in an organic solvent without introducing a base.

In that case, the catalytic entity constituted by the metal M₁ and the activator is desirably prepared in advance.

More precisely, in a first implementation of the invention, using an alcohol with formula (I) carrying a halogenomethylcarbinol group and a hydroxyl group, the oxidation reaction is carried out in an aqueous medium containing, in solution, a basic agent, more particularly ammonium hydroxide, alkali or alkaline-earth bases including hydroxides such as sodium, potassium or lithium hydroxide can be cited; alkali alkanolates such as sodium or potassium methylate, ethylate, isopropylate or tert-butylate, sodium or potassium carbonate or bicarbonate and in general, salts of alkali or alkaline-earth bases and weak acids.

The starting alcohol with formula (I) carries a hydroxyl group which is preferably placed in its salt form before carrying out the oxidation reaction.

For economic reasons, sodium or potassium hydroxide is preferably used. The proportion of mineral base to be used is preferably such that the ratio between the number of OH⁻ moles and the number of moles of compound with formula (I) is between 1 and 2.

The concentration by weight of alcohol with formula (I) in the liquid phase is normally in the range 1% to 40%, preferably in the range 2% to 30%.

In practice, one manner of carrying out the process consists of bringing molecular oxygen of an oxygen-containing gas, for example air, into contact with the solution comprising the alcohol with formula (I), the basic agent, the catalyst based on metal M₁, and the optional activator, in the proportions indicated above.

One preferred implementation of the invention consists firstly of forming the salt of the alcohol with formula (I) prior to the oxidation reaction.

From a practical viewpoint, the alcohol with formula (I) and the basic agent are charged and the compound is obtained in its salt form at ambient temperature (usually between 15° C. and 25° C.).

The catalyst based on metal M₁ and optional activator are then introduced.

The reaction mixture, under a stream of oxygen or an oxygen-containing gas, is then heated to the desired reaction temperature.

In accordance with the invention, the oxidation temperature is preferably selected from a temperature range of 40° C. to 100° C.

Generally, atmospheric pressure is employed, but it is possible to operate at a temperature between 1 and 20 bars.

The mixture is then agitated at the desired temperature until a quantity of oxygen corresponding to that necessary to transform the carbinol group into a carbonyl group has been consumed.

At the end of the reaction, which preferably last 30 minutes to 6 hours, the ketone compound with formula (III) is recovered:

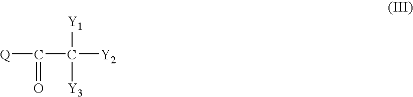

in which formula Q, Y₁, Y₂ and Y₃ have the meanings given above.

After any necessary cooling, the catalytic mass is separated from the reaction mixture, for example by filtering.

In a subsequent step, the medium resulting from adding a protonic acid of mineral origin, preferably hydrochloric acid or sulphuric acid, or an organic acid such as trifluoromethanesulphonic acid or methanesulphonic acid, is acidified to a pH of 5 or less.

The concentration of the acid is not critical and preferably, commercially available forms are used.

Acidification is normally carried out at ambient temperature.

The ketone compound with formula (III) is then recovered using conventional techniques, for example by extraction using a suitable organic solvent, for example a halogenated or non halogenated aromatic hydrocarbon, more particularly toluene or mono- or di-chlorobenzene.

In a further implementation of the invention, a α-halogenated secondary alcohol is used as the starting compound; which is activator of any aliphatic or aromatic type, but which is not phenolic (namely an aromatic compound carrying a hydroxyl group).

In that case, the reaction is advantageously carried out in water or in an organic solvent when the α-halogenated secondary alcohol is not sufficiently soluble in water, for example with a solubility in water at ambient temperature of less than 20% by weight.

An organic solvent which is inert under the reaction conditions and which can at least partially dissolve the starting compound is used.

More specific examples that can be cited are ester type solvents, more particular butyl acetate, amyl acetate and ethyl phthalate.

The concentration of starting substrate in the solvent is preferably in the range 10% to 30% by weight.

As mentioned above, the catalytic entity is preferably prepared extemporaneously.

By way of example, the catalytic entity (M₁ catalyst/activator) can, for example, be prepared by taking a catalyst of a metal M₁ deposited on a support, preferably activated charcoal, silica or alumina, then introducing the compound supplying the activator element, in the presence of a base, preferably sodium carbonate.

This produces the catalyst based on a metal M₁ and an activator.

It is also possible to reduce the catalytic entity with a reducing agent such as hydrogen, formol or hydrazine.

The temperature of the oxidation reaction is preferably selected in a temperature range of 100° C. to 160° C.

In general, atmospheric pressure is used, but it is possible to employ a pressure of 1 to 20 bars.

From a practical viewpoint, the compound with formula (I), the organic solvent and the catalyst are charged.

The reaction medium, kept in a stream of oxygen or an oxygen-containing gas, is then heated to the desired reaction temperature.

The mixture is then stirred at the desired temperature until a quantity of oxygen corresponding to that necessary to transform the carbinol group into a carbonyl group has been consumed.

In an organic medium, the water formed during the reaction is eliminated continuously, by distillation or physically entraining the gas.

At the end of the reaction, which preferably takes 30 minutes to 6 hours, the ketone compound with formula (III) is recovered, usually by distillation.

The invention also provides e-halogenated ketones with general formula:

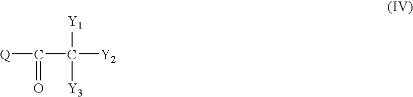

in which formula Q has the meaning given above; preferably, Q represents an aliphatic radical as defined above and Y₁, Y₂ and Y₃ represent a hydrogen atom or a fluorine atom and Y₁, Y₂ and Y₃ represent at least one fluorine atom, preferably three fluorine atoms.

An example of carrying out the invention will now be given by way of non-limiting illustration.

The examples will use the following definitions:

The degree of conversion (TT) corresponds to the ratio between the number of moles of substrate transformed and the number of moles of substrate engaged.

The yield (RR) corresponds to the ratio between the number of moles of product formed and the number of moles of substrate engaged.

The weight of noble metal is expressed as the % by weight with respect to the total weight of catalyst (active phase+support).

### EXAMPLE 1

### Preparation of 4-trifluoroacetylanisole

4 g of 2,2,2-trifluoro-1-(4-methoxybenzene)ethanol was introduced into a 100 ml glass reactor.

40 ml of ethyl acetate was added.

It was stirred, then 0.5 g of 4.7% Pt+1.5% Bi/C catalyst containing 50% water, a product sold by Engelhard, was introduced.

It was heated to 125° C. and a stream of air was passed through the reactor head.

After reacting for 6 hours, the yield (RR) was 99%, determined by gas chromatography.

### EXAMPLE 2

Example 1 was repeated, using a catalyst comprising 5.3% Pd+3% Bi.

After reacting for 6 hours, the yield (RR) was 13%.

### EXAMPLE 3

### Preparation of 4-trifluoroacetyl-2-hydroxytoluene

4 g of 2,2,2-trifluoro-1-(2-hydroxy-3-methylbenzene)ethanol was introduced into a 100 ml glass reactor.

40 ml of water was added.

It was stirred then 0.5 g of a 5.4% Pt+1.8% Bi/C catalyst containing 50% water was introduced.

It was heated to 80° C. and a stream of air was passed into the solution.

After 15 hours under these conditions, the yield (RR) was 95%, determined by gas chromatography.

### EXAMPLE 4

Example 3 was repeated, using 40 ml of butyl acetate.

The reaction was carried out at 125° C.

After reacting for 6 hours, the yield (RR) was 96%.

### EXAMPLE 5

### Preparation of 4-trifluoroacetyl-2-hydroxytoluene

4 g of 2,2,2-trifluoro-1-(2-hydroxy-3-methylbenzene)ethanol was introduced into a 100 ml glass reactor.

40 ml of water and 0.8 g of sodium hydroxide were added.

It was stirred then 0.5 g of a 5% Pt/C catalyst containing 50% water was introduced.

17 mg of Bi₂O₃ was then added.

It was heated to 80° C. and a stream of air was passed into the reaction medium by bubbling.

After 8 hours of reaction, the yield (RR) was 96%.

### EXAMPLE 6

### Preparation of 2,6-trifluoroacetyl-nonyl-4-phenol

8 g of 2,6-(2,2,2-trifluoro-1-ethanol)-4-nonylphenol was introduced into a 100 ml glass reactor.

40 ml of water and 1.3 g of sodium hydroxide pellets were added.

It was stirred then 0.8 g of a 5% Pt/C catalyst containing 50% water and 27 mg of Bi₂O₃ were then added.

It was heated to 80° C. and a stream of air bubbled through.

After 15 hours of reaction, the yield (RR) was 94%.

### EXAMPLE 7

### Preparation of cis and trans 1,1,1-trifluoro-4-nonene-2-one

4 g of cis and trans 1,1,1-hydroxy-2-nonene-4 was introduced into a 100 ml glass reactor.

40 ml of butyl acetate was added and stirring was carried out.

Then 0.5 g of a 4.7% Pt+1.5 Bi/C catalyst containing 50% water was added.

It was heated to 125° C. and a stream of air was passed into the reaction medium.

After 8 hours of reaction, the yield (RR) was 94%.

### EXAMPLE 8

### Preparation of 3-(trifluoroacetyl)pyridine

5 g of (2,2,2-trifluoro-1-ethanol)-3-pyridine was introduced into a 100 ml glass reactor.

40 ml of butyl acetate was added and stirring was carried out.

Then 0.5 g of a 4.7% Pt+1.5 Bi/C catalyst supported on activated charcoal was then added.

It was heated to 125° C. and a stream of air was passed into the reaction medium.

After 10 hours of reaction, the yield (RR) was 68%, obtained by gas chromatography.

1. A process for preparing an α-halogenated ketone, characterized in that it consists of oxidising, in the liquid phase, an α-halogenated secondary alcohol using molecular oxygen or an oxygen-containing gas in the presence of a catalyst based on a metal M₁ selected from metals from groups 1b and 8 of the periodic table.

2. A process according to claim 1, characterized in that the α-halogenated secondary alcohol has general formula (I): 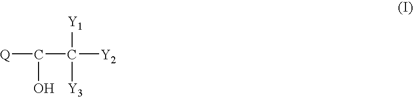
in which formula, Q represents a monovalent hydrocarbon group, optionally substituted, containing 1 to 40 carbon atoms, Y₁, Y₂ and Y₃, which may be identical or different, represent a hydrogen atom or a halogen atom, namely chlorine, fluorine, bromine or iodine, preferably fluorine, or a perhalogenoalkyl group containing 1 to 10 carbon atoms and at least one of groups Y₁, Y₂ and Y₃ represents a halogen atom.

3. A process according to claim 2, characterized in that the α-halogenated secondary alcohol has general formula (I) in which at least two of groups Y₁, Y₂ and Y₃ represent a halogen atom, and more preferably all of groups Y₁, Y₂ and Y₃ represent a halogen atom, preferably a fluorine atom.

4. A process according to claim 2 or claim 3, characterized in that the α-halogenated secondary alcohol has general formula (I) in which in which Q represents a monovalent hydrocarbon group, which may or may not be substituted, which may be a linear or branched, saturated or unsaturated acyclic aliphatic group; or a saturated, unsaturated or aromatic, monocyclic or polycyclic carbocyclic or heterocyclic group.

5. A process according to any one of claims 2 to 4, characterized in that the α-halogenated secondary alcohol has general formula (I) in which Q represents an aryl group with general formula (II): 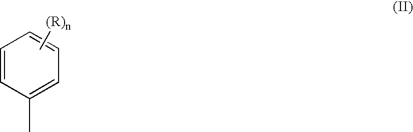
in which formula (II):
n is a whole number from 0 to 5, preferably 0 to 3;
R represents R₁, one of the following groups or functions:
a linear or branched alkyl group containing 1 to 6 carbon atoms, preferably 1 to 4 carbon atoms, such as methyl, ethyl, propyl, isopropyl, butyl, isobutyl, sec-butyl or tert-butyl;
a halogenoalkyl group containing 1 to 6 carbon atoms, which may be mono-, poly- or perhalogenoalkyl and containing 1 to 13 halogen atoms;
a linear or branched alkenyl group containing 2 to 6 carbon atoms, preferably 2 to 4 carbon atoms, such as vinyl or allyl;
a linear or branched alkoxy or thioether group containing 1 to 6 carbon atoms, preferably 1 to 4 carbon atoms, such as methoxy, ethoxy, propoxy, isopropoxy or butoxy;
a group with formula:
—OH
—COOH
—CHO
—CN
—N—(R₂)₂
—X
—CF₃
in which formulae groups R₂, which may be identical or different, represent a hydrogen atom, a linear or branched alkyl group containing 1 to 6 carbon atoms, more preferably 1 to 4 carbon atoms, or a phenyl group and X represents a halogen atom, in particular a chlorine or bromine atom;
R represents R₃, one of the following more complex groups:
a group: 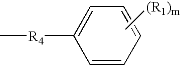
in which:
R₁ has the meaning given above;
R₄ represents a covalent bond or a linear or branched, saturated or unsaturated divalent hydrocarbon group containing 1 to 4 carbon atoms, such as methylene, ethylene, propylene, isopropylene or isopropylidene;
and m is a whole number from 0 to 3;
a group R₄—A—R₅, in which:
R₄ has the meaning given above;
R₅ represents a linear or branched alkyl group containing 1 to 6 carbon atoms, preferably 1 to 4 carbon atoms, or a group: 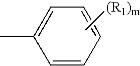
and A one of the following groups: 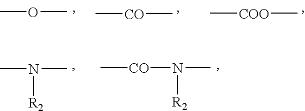
in which formulae m, R₁ and R₂ have the meanings given above.

6. A process according to claim 5, characterized in that the α-halogenated secondary alcohol has general formula (I) in which Q represents an aryl group with general formula (II) in which n is more than 1 and groups R are identical or different, and two successive carbon atoms of the benzene ring are bonded together via a ketal bridge, preferably by an extranuclear methylene or ethylene group.

7. A process according to claim 5 or claim 6, characterized in that the α-halogenated secondary alcohol has general formula (I) in which Q represents an aryl group with general formula (II) in which:
R represents one of the following functional groups:
a linear or branched alkyl group containing 1 to 4 carbon atoms;
a linear or branched alkoxy or thioether group containing 1 to 4 carbon atoms;
a methylene or ethylene-dioxy group;
an —OH group;
a phenyl or benzyl group;
a halogen atom.

8. A process according to any one of claims 2 to 4, characterized in that the α-halogenated secondary alcohol has formula (I) in which Q represents a carbocyclic group that is saturated or comprises 1 or 2 unsaturated bonds in the cycle, generally containing 3 to 7 carbon atoms, preferably 6 carbon atoms in the cycle; said cycle preferably being substituted by 1 to 5 groups R₁, preferably 1 to 3, R₁ having the meanings given above in claim 5.

9. A process according to any one of claims 2 to 4, characterized in that the α-halogenated secondary alcohol has general formula (I) in which Q represents a linear or branched alkyl, alkenyl, alkadienyl or alkynyl group, preferably containing 1 to 12 carbon atoms; the hydrocarbon chain can optionally be:
interrupted by one of the following groups: 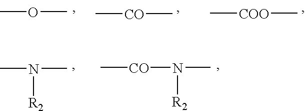
in which formulae R₂ has the meanings given above in claim 5;
and/or carries one of the following substituents:
—OH, —COOH, —CHO, —CN, —N(R₂)₂, —X, —CF₃
in which formulae R₂ has the meanings given above in claim 5.

10. A process according to claim 9, characterized in that the α-halogenated secondary alcohol has general formula (I) in which Q represents a linear or branched, saturated or unsaturated acyclic aliphatic group carrying a cyclic substituent, said acyclic aliphatic group possibly being bonded to the cycle via a covalent bond or via one of the following groups:
—OH, —COOH, —CHO, —CN, —N(R₂)₂, —X, —CF₃
in which formulae R₂ has the meanings given above in claim 5.

11. A process according to claim 10, characterized in that the α-halogenated secondary alcohol has general formula (I) in which Q represents a linear or branched, saturated or unsaturated acyclic aliphatic group carrying a cycloaliphatic, aromatic or heterocylic substituent optionally carrying 1 to 5 groups R₁, which may be identical or different, as defined in claim 5.

12. A process according to any one of claims 2 to 4, characterized in that the α-halogenated secondary alcohol has general formula (I) in which Q represents a saturated or non saturated monovalent heterocyclic group, in particular containing 5 or 6 atoms in the cycle, 1 or 2 heteroatoms of which are atoms such as nitrogen, sulphur or oxygen, the carbon atoms of the heterocycle possibly being either completely or partially substituted by groups R₁, R₁ having the meanings given above in claim 5.

13. A process according to any one of claims 1 to 12, characterized in that the α-halogenated secondary alcohol is 2-hydroxy-1-phenyl-trichloromethylcarbinol, 2-hydroxy-1-phenyl-trifluoromethylcarbinol, 4-hydroxy-1-phenyl-trichloromethylcarbinol, 4-hydroxy-1-phenyl-trifluoromethylcarbinol, 3-hydroxy-4-methoxy-1-phenyl-trichloromethylcarbinol, 3-hydroxy-4-methoxy-1-phenyl-trifluoromethylcarbinol, 4-hydroxy-3-methoxy-1-phenyl-trichloromethylcarbinol, 4-hydroxy-3-methoxy-1-phenyl-trifluoromethylcarbinol, 3,4-dihydroxy-1-phenyl-trichloromethylcarbinol, 3,4-dihydroxy-1-phenyl-trifluoromethylcarbinol, 3,5-dihydroxy-1-phenyl-trichloromethylcarbinol, 3,5-dihydroxy-1-phenyl-trifluoromethylcarbinol, 3,5-dihydroxy-4-methyl-1-phenyl-trichloromethylcarbinol, 3,5-dihydroxy-4-methyl-1-phenyl-trifluoromethylcarbinol, 3,4-dimethoxy-1-phenyl-trifluoromethylcarbinol, 3,4-methylenedioxy-1-phenyl-trifluoromethylcarbinol, 1,1,1-trifluoro-4-octene-2-ol or 1,1,1-trifluoro-4-decene-2-ol.

14. A process according to claim 1, characterized in that an activator for metals from groups 1b and 8 is used, such as cadmium, cerium, bismuth, lead, silver, tellurium, tin or germanium, preferably bismuth.

15. A process according to claim 1, characterized in that the catalyst is based on copper, nickel, ruthenium, rhodium, palladium, osmium, iridium, platinum or mixtures thereof; said catalyst preferably being based on platinum and/or palladium.

16. A process according to claim 15, characterized in that the platinum and/or palladium catalyst is supplied in the form of platinum black, palladium black, platinum oxide, palladium oxide or the noble metal itself deposited on various supports such as carbon black, calcium carbonate, activated alumina and silica or similar substances, preferably carbon black.

17. A process according to claim 1, characterized in that the quantity of catalyst to be used, expressed as the weight of metal M₁ with respect to that of the compound with formula (I), is between 0.01% and 10%, preferably 0.04% to 2%.

18. A process according to claim 14, characterized in that the activator is an organic or inorganic bismuth derivative selected from the group formed by: bismuth oxides; bismuth hydroxides; bismuth or bismuthyl salts of mineral hydrogen acids, preferably chloride, bromide, iodide, sulphide, selenide or telluride; bismuth or bismuthyl salts of mineral oxyacids, preferably sulphite, sulphate, nitrite, nitrate, phosphite, phosphate, pyrophosphate, carbonate, perchlorate, antimonate, arsenate, selenite or selenate; bismuth or bismuthyl salts of aliphatic or aromatic organic acids, preferably acetate, propionate, salicylate, benzoate, oxalate, tartrate, lactate or citrate; and bismuth or bismuthyl phenates, preferably gallate or pyrogallate.

19. A process according to claim 18, characterized in that the bismuth derivative is selected from the group formed by bismuth oxides Bi₂O₃ and Bi₂O₄; bismuth hydroxide Bi(OH)₃, bismuth chloride BiCl₃; bismuth bromide BiBr₃; bismuth iodide BiI₃; neutral bismuth sulphate Bi₂(SO₄)₃; neutral bismuth nitrate Bi(NO₃)₃,5H₂O; bismuthyl nitrate (BiO)NO₃; bismuthyl carbonate (BiO)₂CO₃,0.5H₂O; bismuth acetate Bi(C₂H₃O₂)₃; and bismuthyl salicylate C₆H₄CO₂(BiO)(OH).

20. A process according to claim 14, characterized in that the quantity of activator, expressed with respect to the weight of metal M₁ employed, is in the range 0.1% to 100%, preferably about 50%.

21. A process according to claim 1, characterized in that oxidation is carried out in an aqueous medium containing a basic agent when the α-trihalogenated secondary alcohol with formula (I) is an aromatic compound with formula (I) carrying a halogenomethylcarbinol group and a hydroxyl group on its cycle.

22. A process according to claim 21, characterized in that the basic agent is sodium hydroxide.

23. A process according to claim 21, characterized in that the salt form of the alcohol with formula (I) is formed before the oxidation reaction.

24. A process according to any one of claims 21 to 23, characterized in that the alcohol with formula (I) and the basic agent are charged and the compound is obtained in its salt form at ambient temperature, then the catalyst based on a metal M₁ is introduced with the optional activator, and the reaction mixture, maintained in a stream of oxygen or an oxygen-containing gas, is then heated to the desired reaction temperature.

25. A process according to any one of claims 21 to 24, characterized in that the oxidation reaction is carried out in a temperature range of 40° C. to 100° C.

26. A process according to claim 1, characterized in that oxidation is carried out in an aqueous or organic medium when the α-halogenated secondary alcohol with formula (I) is any α-halogenated secondary alcohol with the exception of aromatic compounds carrying a halogenomethylcarbinol and an hydroxyl group on the cycle.

27. A process according to claim 26, characterized in that the organic solvent is of the ester type, preferably butyl acetate.

28. A process according to claim 26, characterized in that the catalytic entity comprising the metal M₁ and the activator is prepared in advance.

29. A process according to any one of claims 26 to 28, characterized in that the temperature of the oxidation reaction is between 100° C. and 160° C.

30. A process according to any one of claims 26 to 29, characterized in that the compound with formula (I), water or the organic solvent and the catalytic entity are charged together.

31. α-halogenated ketones with general formula: 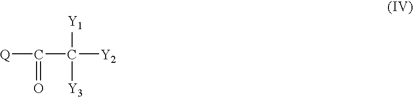
in which formula, Q represents a radical as defined in any one of claims 4 to 12 and Y₁, Y₂ and Y₃ represent a hydrogen atom or a fluorine atom and Y₁, Y₂ and Y₃ represent at least one fluorine atom, preferably three fluorine atoms.

32. A ketone according to claim 31, characterized in that it has formula (IV) in which Q represents an aliphatic radical as defined in claim 9 and Y₁, Y₂ and Y₃ represent a hydrogen atom or a fluorine atom and Y₁, Y₂ and Y₃ represent at least one fluorine atom, preferably three fluorine atoms.

33. 1,1,1-trifluoro-4-octene-2-one;
1,1,1-trifluoro-4-nonene-2-one;
1,1,1-trifluoro-4-decene-2-one;
4-trifluoroacetylanisole;
4-trifluoroacetyl-2-hydroxytoluene;
2,6-trifluoroacetyl-nonyl-4-phenol;
3-(trifluoroacetyl)pyridine.
